(19) United States
(12) Patent Application Publication
(10) Pub. No.: US 2024/0325008 A1
(43) Pub. Date: Oct. 3, 2024

(54) A MULTIFUNCTIONAL DEVICE FOR MINIMALLY INVASIVE SURGERIES AND A METHOD THEREOF

(71) Applicant: DENOVO BIOINNOVATIONS PRIVATE LIMITED, Pune (IN)

(72) Inventors: Nikhil MAMORIA, Pune (IN); Himanshu LADDHA, Pune (IN)

(21) Appl. No.: 18/699,993

(22) PCT Filed: Oct. 14, 2022
(86) PCT No.: PCT/IN2022/050917

§ 371 Date: Apr. 10, 2024

(30) Foreign Application Priority Data
Oct. 14, 2021 (IN) 202121046917

(51) Int. Cl. A61B 17/00; A61B 17/04; A61B 17/32; A61B 17/3201

(52) U.S. Cl. CPC A61B 17/00234; A61B 17/0469; A61B 17/3201; A61B 2017/00022; A61B 2017/00119; A61B 2017/00353; A61B 2017/00371; A61B 2017/00398; A61B 2017/00411; A61B 2017/00438; A61B 2017/00477; A61B 2017/047; A61B 2017/0474; A61B 2017/320064

(57) ABSTRACT

Multifunctional device for MIS includes handle 200, a shaft 700, a multifunctional head assembly 300 and an articulation joint 800. The head assembly 300 includes a suturing unit 1100/1400 and an upper and a lower grasper unit 400 located in planes above and below the suturing unit. Each grasper unit 400n comprises a pair of graspers 402 configured to pivotally move in the plane of the grasper unit 400 about axis 414 to grasp tissues or a suturing thread, as well as to move pivotally perpendicular to the plane of the grasper unit 400 about axis 412 to pull the grasped suturing thread for knot tightening. Configuration of the head assembly 300 causes loose end portion of thread, when grasped by grasper unit 400, to be located within a circular zone of needle, which enables looping of needle side portion of thread around loose end portion.

### TECHNICAL FIELD

The present disclosure relates to the field of surgical instruments. In particular, the present disclosure relates to a device for minimally invasive surgeries that includes functionalities of grasping, suturing, knotting, knot tightening, cutting and cautery during surgeries.

### BACKGROUND

Background description includes information that may be useful in understanding the present invention. It is not an admission that any of the information provided herein is prior art or relevant to the presently claimed invention, or that any publication specifically or implicitly referenced is prior art.

One of the most important advances in surgical technology in the last two decades of the twentieth century is represented by the introduction of minimally Invasive Surgery (MIS).

Minimally Invasive Surgery, commonly known as Laparoscopic or Endoscopic surgery, is performed by making 4 to 5 small incisions of 5-12 mm size on the abdomen of the patient for inserting different devices/instruments and accessing the point of surgery. Entire surgical procedures are performed by manipulating the tool handles from outside the patient's body, without direct contact with the diseased tissue or direct vision of the operative site. The operative field is viewed by a laparoscope(miniature camera) also inserted into the abdominal cavity through one of the incisions in the abdominal wall. The laparoscopic view of the operative site is captured by a miniature camera in the laparoscope and displayed on a 2-D video monitor for the operating surgeon.

With the gradual replacement of the conventional open-cut approach surgery with the MIS techniques patient benefits like smaller tissue trauma, smaller scar, minimal blood loss, less pain, faster recovery time, and early hospital discharge has remained a consistent finding. Minimally Invasive Surgery (MIS) has become commonplace for an ever-growing number of surgical procedures and can replace almost all open abdominal surgical procedures.

Laparoscopy is a technological marvel which resolved all the patient concerns but raised the demands placed on surgeons without the necessary instruments required to do it. Despite advancements in surgical techniques and the availability of newer surgical tools, the complexity of laparoscopic suturing and knotting continues to be a barrier to greater adoption of MIS procedures, which deprives the patients of potential benefits of laparoscopy.

During MIS, a surgeon is confronted with the technical challenge of performing all maneuvers using long rigid instruments. For each use, the surgeon has to use a specific instrument and has to keep interchanging the instruments during the whole surgical procedure. For grasping laparoscopic graspers are used, for cutting tissue laparoscopic scissors are used, for cauterization of tissue surgeon has to insert cautery and use a foot paddle to operate the cautery. For intracorporeal suturing surgeons has to insert a needle inside the abdomen first and then use laparoscopic graspers and needle holder to complete the suturing. Due to this all of the surgeon's hands and feet are occupied by different instruments.

Most of the surgical time is spent interchanging various instruments which affects the surgeon's focus as they have to continuously change instruments. This increase in the operating time increases the use of anesthesia on the patient, thus making him prone to complications due to prolonged anesthesia.

Also, the tissue is not in direct contact with the surgeon's hands, and because of 2D vision surgeons lack the perception of the depth and the thickness of tissue. Without necessary experience, the increased force can result in tissue crush injuries.

Laparoscopic intracorporeal suturing means tissue approximation within the body using suture material. One laparoscopic needle holder & grasper and a needle with suture thread are used to tie a series of knots inside the body. This technique requires the skill to manipulate the needle, pass it from 1 needle holder to the next, and execute a series of knots.

Laparoscopic intracorporeal suturing is the preferred method because it is highly adaptable, flexible, economical, and uses commercially available instruments. Although Laparoscopic suturing is the most basic but most challenging to perform and learn. It is considered a significant barrier to the broader adoption of laparoscopic surgery. It is done using a suture needle, laparoscopic needle holder, and grasper.

Intracorporeal suturing is viewed as a recurring cycle of events: starting with grasping the tissue using graspers, grasping the suture needle with a needleholder, driving the needle across the anastomosis edge of the tissue, re-adjusting the needle holder, grasping the other edge of tissue driving the needle across the other anastomosis edge, tying three knots, pulling both the ends of the suture thread to tighten the knot and ending with thread cutting.

Ideally, the needle should be oriented such that the directions of (1) the needle tip and (2) the needle-driving force must be identical; the optimal direction for both is 90°, head-on against tissue resistance. Tissue should be grasped before the suturing to create tension in tissue so the needle can pierce easily without much resistance. If tissue is not grasped and the needle is pierced it may result in tissue tear and the path of the needle can change.

Intracorporeal suturing requires repetitive dual-hand-coordinated movements in an environment with a limited degree of freedom for each step of suturing. During suturing, the surgeon needs to constantly change the orientation of the needle to find the appropriate pose. To align the needle accurately successive grasp and release operation is required. Due to the structure of the standard needle holder, the orientation of the needle during the intracorporeal suturing is not completely controllable and multiple grasps and release movements are required to position the needle before the execution of each stitch. This requires training and can result in extended surgical time. Without necessary experience and practice, these constraints increase the surgeons' effort which leads to physical fatigue resulting in an increased level of errors and poor suturing quality, while mental stress results in increased operating time and anesthesia used. These factors make laparoscopy ergonomically more demanding.

Currently, there is no device in the market that can assist surgeons with all of the aforementioned functions using a single instrument.

One promising solution is Single-incision laparoscopy surgery (SILS), which is a type of minimally invasive surgical procedure where only 1 incision is made and multiple instruments are inserted through this single incision. This development was mainly done to reduce the number of incisions on the abdomen and for early recovery. Despite this development, SILS is not commonly performed because it adds to the complexity of the surgery as the surgeon has to insert all the instruments through a single port and repetitively interchange them for the desired function. In view of the above, the conventional devices are outdated and inefficient because the endpoint of the instrument moves in the opposite direction of the surgeon's hands, leading to very tiring manipulation since the surgeon has to do needle positioning and orientation under conditions of poor needle control and a limited degree of freedom. The limitations make the process very tedious and exhaustive process requiring a steep learning curve.

Therefore, there is a requirement for an improved laparoscopic surgical device that can facilitate grasping, cutting, cauterizing, suturing, and knotting, among others, during laparoscopic surgery overcoming the limitations of conventional laparoscopic surgical devices.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### OBJECTS OF THE INVENTION

A general object of the present disclosure is to overcome limitations of the conventional surgical devices.

An object of the present disclosure is to provide a single device for laparoscopic surgery that facilitates grasping, cutting, cauterizing, suturing, knotting and knot tightening, among others, during laparoscopic surgery.

Another object of the present disclosure is to provide a laparoscopic surgical device that can perform most laparoscopic functions using a single instrument, thereby eliminating the need to switch instruments for each function.

Another object of the present disclosure is to provide a laparoscopic surgical device that includes feedback-based grasping of tissues allowing control of grasping force.

Another object of the present disclosure is to provide a laparoscopic surgical device that includes mechanism for detecting the presence of a blood vessel in the grasped tissues to prevent accidental damage to the blood vessel.

Another object of the present disclosure is to provide a laparoscopic surgical device that incorporates 6 degrees of freedom in space for movements of a head assembly.

Another object of the present disclosure is to provide a laparoscopic surgical device that automates the whole process of intracorporeal suturing, knotting, and knot tightening into a single process.

Another object of the present disclosure is to provide a laparoscopic surgical device that avoids multiple grasps and release movements of a needle during the process of intracorporeal suturing.

Another object of the present disclosure is to provide a laparoscopic surgical device that can be easily incorporated with surgical robotics systems.

Another object of the present disclosure is to provide a laparoscopic device which can assist the surgeons in single incision laparoscopic surgeries.

The other objects and advantages of the present invention will be apparent from the following description when read in conjunction with the accompanying drawings, which are incorporated for illustration of the preferred embodiments of the present invention and are not intended to limit the scope thereof.

### SUMMARY

Aspects of the present disclosure relate to a surgical instrument. In particular, it relates to a device minimally invasive surgery (also referred to as a laparoscopic surgical device, hereinafter), incorporating the functions/mechanisms of grasping, suturing, knotting, knot tightening, tissue cutting, and cauterization.

In an embodiment, the proposed device for minimally invasive surgery includes a handle; a shaft coupled to a distal end of the handle, and a multifunctional head assembly configured at the distal end of the shaft through an articulation joint such that the articulation joint allows the head assembly to pivotally move relative to the shaft in two degrees of freedom. The head assembly includes a suturing unit and a pair of grasper units comprising an upper grasper unit located in a plane above the suturing unit, and a lower grasper unit located in a plane below the suturing unit. Each of the upper and lower grasper units comprises a pair of graspers that are configured to pivotally move in the plane of the corresponding grasper unit to grasp tissues or a suturing thread, as well as to move pivotally perpendicular to the plane of the grasper unit to pull the grasped suturing thread for knot tightening.

The graspers of at least one of the upper and lower grasper units can include a cutting blade configured within the grasper for sliding movement such that in an actuated position, a cutting edge of the cutting blade projects out of a slit on a grasping face of the grasper for use of the grasper unit as scissors.

The shaft may include an outer tubular casing and an inner tube coupled to the head assembly through the articulation joint. The inner tube may be coupled to a rotating knob located at a distal end of the handle such that the rotation of the rotating knob by a user results in rotation of the head assembly about a longitudinal axis of the shaft.

The articulation joint may be coupled to a manipulator provided on the handle through a pair of manipulator ropes. The manipulator may be located on the handle such that a joystick of the manipulator is operable by a thumb of a user. Movement of the joystick laterally and in up and down direction results in the articulation joint pivotally moving the head assembly about the two mutually perpendicular axes, each perpendicular to the longitudinal axis of the shaft, thereby providing three degrees of rotational freedom to the head assembly.

The manipulator may include an upper gimbal and a lower gimbal placed over one another and coupled with a joystick for pivotal motion about two mutually perpendicular axes. Each gimbal may be coupled to a pulley to pull respective manipulator ropes that couple the manipulator to the articulation joint.

The manipulator may include a lock button that is configured in combination with the joystick to press a set of stoppers against the gimbals to force the gimbals against each other to lock the manipulator in any position. Pressing the lock button again separates the stoppers from the gimbals to allow movement of the gimbals for manipulating the articulation joint.

Each of the graspers may include a front body and a rear body. The front body may be pivotally coupled to the rear body for rotation about an axis that is parallel to the plane of the corresponding grasper unit. The rear body may be coupled to a pinion such that rotation of the pinion results in pivotal movement of the grasper about an axis perpendicular to the plane of the corresponding grasper unit,

Each of the grasper units may include a rack in engagement with the pinions of the corresponding graspers such that pulling of the rack towards the proximal side results in the two graspers to move towards each other for grasping a tissue or a suturing thread. The racks of each of the grasper units may be coupled to a corresponding pulley in the handle by a grasper rope such that rotation of the pulley causes the grasper rope to be pulled to move the graspers to grasp a tissue or a suturing thread. The movement of the rack may be controlled based on feedback such that force of closing of graspers is controlled.

The pulleys may be coupled to a corresponding bevel gear and a grasper activation knob, wherein the grasper activation knobs have a cam mechanism such that a first pressing of the grasper activation knob causes the corresponding bevel gear to move inwards to engage with the input bevel gear coupled to a motor, and a second pressing of the grasper activation knob results in the bevel gear to move outwards to disengage from the input bevel gear.

The grasper activation knobs may include an inbuilt locking mechanism to lock the corresponding pulley, and thereby the corresponding grasper unit in an actuated position, and wherein the locking mechanism is configured such that, pulling the grasper activation knob outwards unlocks the pulley and the grasper unit to return the graspers to an open position under a biasing force.

In another embodiment of the multifunctional device for minimally invasive surgery, the device includes a handle, a shaft coupled to a distal end of the handle, and a head assembly configured at a distal end of the shaft. The head assembly includes at least one grasper unit having a pair of graspers to grasp at least a tissue. Each of the graspers of the at least one grasper unit includes a cutting blade configured within the grasper for sliding movement such that in an actuated position a cutting edge of the cutting blade projects out of a slit on a grasping face of the grasper for use of the grasper unit as a scissors. The blades may be configured to get flow of any of a direct and alternating current for the blades to function as cautery.

In another embodiment of the multifunctional device for minimally invasive surgery, the device includes a handle, a shaft coupled to a distal end of the handle and a head assembly configured at the distal end of the shaft. The head assembly includes at least one grasper unit, the grasper unit having a pair of graspers to grasp at least a tissue. The grasper units include a set of infrared sensors and pressure sensors configured on the graspers to provide feedback on the thickness of the tissue being grasped and pressure applied on the tissue during grasping to control the actuation of the grasper units to prevent crushing of the grasped tissue.

In another embodiment of the multifunctional device for minimally invasive surgery, the device includes a handle, a shaft coupled to a distal end of the handle and a head assembly configured at the distal end of the shaft. The head assembly includes at least one grasper unit, the grasper unit having a pair of graspers to grasp at least a tissue. The at least one grasper unit includes a set of infrared sensors configured on the graspers, and the device includes a controller to detect the presence of a blood vessel in the grasped tissue based on pulsation in the signal from the infrared sensors, the signal being indicative of presence of blood vessel in the grasped tissue. The controller is further configured to issue a warning to a user of the device if a blood vessel is detected.

An embodiment of the present disclosure provides a method for suturing, knotting and knot tightening a tissue during a minimally invasive surgery, the method including the steps of: providing a device for minimally invasive surgery, the device having: a suturing unit; an upper grasper unit located in a plane above the suturing unit; and a lower grasper unit located in a plane below the suturing unit. Graspers of each of the upper and lower grasper units are configured to pivotally move in the plane of the corresponding grasper unit, as well as to move pivotally perpendicular to the plane of the grasper unit to move away from the suturing unit. The method further includes the steps of: grasping the tissue by an upper grasper unit and a lower grasper unit by moving the corresponding graspers in the planes of the respective grasper unit; biting the grasped tissue by a needle of the suturing unit to cause a suturing thread fixed to the suturing needle to penetrate and cross the tissue; releasing the grasped tissue; grasping a free end portion of the suturing thread by one of the upper or lower grasper units such that the free end portion of the suturing thread is located within a circular zone defined by movement of the needle; moving the needle at least by 360 degrees to wind the suturing thread around the free end portion of the suturing thread to take one loop and repeat according to type of knot; grasping a needle side portion of the suturing thread by other of the upper and lower grasper units; and moving the graspers of the upper and lower grasper units perpendicular to the plane of the respective grasper units to tighten a knot.

In still another embodiment, the present disclosure provides a suturing unit for minimally invasive surgery, having a housing having a pair of more than half circle tracks comprising an upper track and a lower track, and a needle track; a suturing needle positioned in the needle track for movement along the needle track; and a puck in engagement with the upper track or the lower track at a given time. The puck, when in engagement with the lower track presses against the suturing needle such that movement of the puck results in movement of the suturing needle, and when in engagement with the upper track, the puck is lifted off the suturing needle and moves without imparting movement to the suturing needle. The suturing unit further includes a driving mechanism to move the puck along the upper track and the lower track. The upper track and the lower track are connected to each other at their two ends such that when the puck is moved to the end of the lower track, the puck shifts upward to get engaged to the upper track, and when the puck is moved in reverse direction to the end of the upper track the puck shifts downward to get engaged to the lower track.

The driving mechanism of the suturing unit can be a scotch yoke mechanism, and the upper track, lower track and the needle track may be configured on any of an inner periphery and an outer periphery of the housing.

In another embodiment of the suturing unit for minimally invasive surgery, the suturing unit includes a housing having a needle track configured on an outer periphery of the housing; a suturing needle positioned in the needle track for movement along the needle track; and a belt configured around the outer periphery of the housing such that the belt presses against the suturing needle to move the needle along the needle track as the belt moves. A plurality of pulleys are arranged to move the belt around the outer periphery of the housing. One of the plurality of pulleys is coupled to a motor by a flexible rotary shaft for driving the belt.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIGS. 1A-1C illustrate exemplary left side view, right side view and top view respectively of the disclosed device for minimally invasive surgery, in accordance with embodiments of the present disclosure.

FIG. 2 illustrates an exemplary transparent perspective view of a handle of the device showing different components therein, in accordance with embodiments of the present disclosure.

FIG. 3 illustrates an exemplary exploded view of a head assembly of the device, in accordance with embodiments of the present disclosure.

FIG. 4A illustrates an exemplary exploded view of a grasper unit of the head assembly of the device, in accordance with embodiments of the present disclosure.

FIG. 4B illustrates exemplary top views of a grasper unit of the head assembly of the device, showing grasping of a tissue by pair of graspers, in accordance with embodiments of the present disclosure.

FIGS. 5A to 5C illustrate exemplary views of a grasper of the grasper unit showing features that enable scissors functionality of the grasper unit, in accordance with an embodiment of the present disclosure.

FIG. 6 illustrates an exemplary block diagram of the device showing different elements that enable different functionalities of the head assembly, in accordance with embodiments of the present disclosure.

FIGS. 7A and 7B illustrates exemplary views of a shaft of the device, in accordance with embodiments of the present disclosure

FIGS. 8A and 8B illustrate exemplary top view and perspective view respectively of an articulation joint that couples the head assembly to the shaft, in accordance with embodiments of the present disclosure.

FIG. 9 illustrates an exemplary exploded view of a manipulator of the device, in accordance with embodiments of the present disclosure.

FIG. 10 illustrates an exemplary perspective view of an arrangement for coupling a motor to driving mechanisms of different grasper units and a knot tightening activation knob of the device, in accordance with embodiments of the present disclosure.

FIGS. 11A to 11C illustrate details of a suturing unit of the device, in accordance with embodiments of the present disclosure.

FIG. 12 illustrates an exemplary view of a suturing needle used with the suturing unit, in accordance with embodiments of the present disclosure.

FIGS. 13A to 13D illustrate functioning of a suturing unit, in accordance with embodiments of the present disclosure.

FIGS. 14A and 14B illustrate exemplary exploded views of an alternate embodiment of the suturing unit.

FIGS. 15A and 15B illustrate exemplary views of the head assembly showing knotting and knot tightening, in accordance with embodiments of the present disclosure.

FIG. 16 is an exemplary method flow diagram for the method for suturing, knotting and knot tightening using the head assembly of the disclosed device, in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Various terms are used herein. To the extent a term used in a claim is not defined, it should be given the broadest definition persons in the pertinent art have given that term as reflected in printed publications and issued patents at the time of filing.

Embodiments described herein relate to a device for minimally invasive surgeries that is multifunctional having capability to carry out grasping, cutting, cauterizing, suturing, knotting and knot tightening. The device is based on a unique multifunctional head assembly that includes a suturing unit and a pair of grasper units, comprising an upper grasper unit located in a plane above the suturing unit, and a lower grasper unit located in a plane below the suturing unit. Each of the grasper units includes a pair of graspers that have capability to pivotally move about two different axes, one in the plane of the grasper unit for movement of the graspers perpendicular to the plane, and other axis that is perpendicular to the plane for pivotal rotation of the grasper perpendicular to the plane of the grasper unit.

Movement of the graspers in the plane of the grasper unit enables grasping of tissues and suturing thread, and movement perpendicular to the plane of the grasper unit enable pulling of the grasped thread for knot tightening.

It is to be appreciated that though the disclosed device has been described with a number of structural features and functionalities, it is possible to configure the device in different configurations having different combinations of the features and functionalities depending on necessity and application, and all such variations are well within the scope of the present disclosure without any limitations whatsoever.

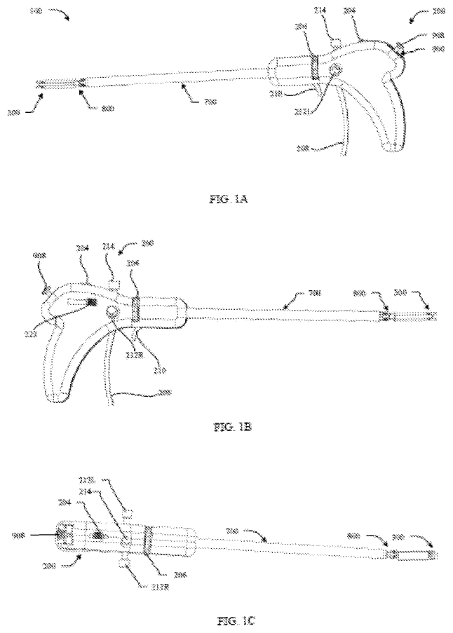

Referring now to FIGS. 1A-C where views of the disclosed laparoscopic surgical device from the left side, right side and top respectively, are disclosed, the device 100 can include a handle 200, a shaft 700 coupled to the handle 200 at a distal end of the handle 200, a multifunctional head assembly 300 coupled to a distal end of the shaft 700 by an articulation joint 800.

The articulation joint 800 can allow the head assembly 300 to be pivotally moved, using the manipulator 900 provided on the handle 200, about two mutually perpendicular axes, each axis perpendicular to a longitudinal axis of the shaft 700. The head assembly can also be rotated about the longitudinal axis of the shaft 700 by rotating a rotating knob 206 provided on the handle 200. Thus the head assembly has 3 degrees of rotational freedom to help a surgeon to manipulate the head assembly 300 remotely, by using the manipulator 900 and the rotating knob 206, during laparoscopic surgery.

The diameter of the head assembly 300, the articulation joint 800 and the shaft 700 can be in the range of 8-12 mm, and the length of the device can be in the range of 250 mm to 400 mm, to be selected depending on nature of the surgery, thereby being suitable for all types of minimally invasive surgical procedures. It can also be used in other types of surgical procedures with suitable modifications that would be obvious to those skilled in the art.

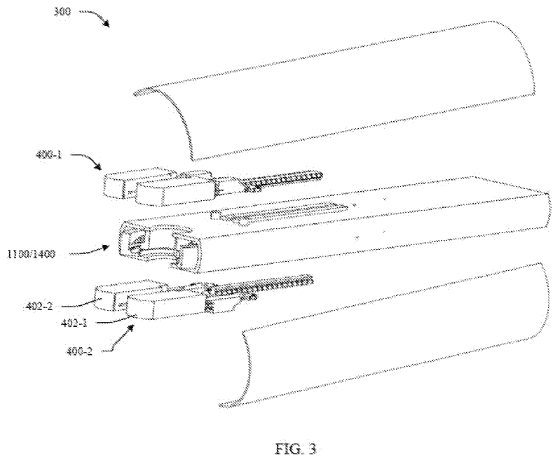

The head assembly 300 incorporates a suturing unit, an upper grasper unit located in a plane above the suturing unit; and a lower grasper unit located in a plane below the suturing unit, as shown in FIG. 3. By virtue of having two grasper units, one on each side of the suturing unit, and by virtue of having graspers in the grasper units that pivotally move in the plane of the corresponding grasper unit, as well as move pivotally perpendicular to the plane of the grasper unit, the device 100 enables a unique way of processing multiple steps of a surgical procedure, like grasping of a tissue, suturing, knotting, knot tightening, cutting, and cauterization, all being achieved using a single handheld device, wherein the function can be switched using a function shifter button 204 provided on the handle 200. Thus, use of multiple devices and multiple grasps and release movements during the process of intracorporeal surgical procedures, which has been the norm so far, is avoided making the intracorporeal surgical procedures more efficient.

The device also provides the surgeon easier access to reach complex anatomical location by 6 degrees of freedom (DoF), which include three DoF as described above plus longitudinal linear movement of the shaft 700 and two linear movements, including lateral and up—down movements of the head assembly 300 by manipulation of the complete device 100. The surgeon can pivotally move the head assembly using a joystick 908 of the manipulator 900 on the handle. The joystick 908 can be moved in 4 directions i.e., right, left, up, and down in the corresponding slots. When the joystick 908 is moved to the right, the head assembly 300 moves to the right and vice versa. Similarly, when the joystick 908 is moved upwards the head assembly 300 moves upward and vice versa. The joystick 909 is positioned such that it can be accessed by a thumb of the surgeon. The rotating knob 206 can rotate the head assembly to 360 degrees about the longitudinal axis of the shaft 700.

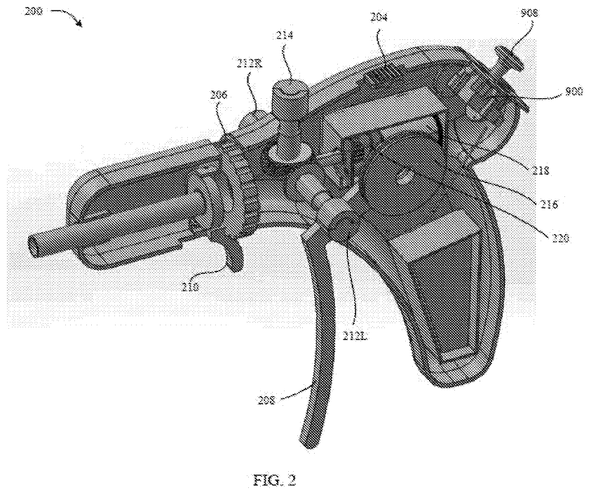

FIG. 2, with reference to FIGS. 1A-1C, shows further details of the handle 200. The handle 200 has the ergonomics of a gun so that it makes easier for the surgeons to grab the handle and easily operate it. The handle has five surfaces, comprising a top surface, a bottom surface, a back surface, a right surface and a left surface. The top surface of the handle 200 includes a knot tightening activation knob 214 and the function shifter button 204. The top surface also contains the rotating knob 206 for rotating the head assembly 300. The knot tightening activation knob 214 can be used for activating knot tightening. The function shifter button 204 can be a sliding switch, which can be used to switch the function between grasping, cutting, knot tightening, cautery and suturing.

The back surface of the handle 200 joining the top surface contains the joystick 908 of the manipulator 900 for manipulating the head assembly 300 by pivotally moving the head assembly 300 up-down and left-right. The manipulator 900 includes a locking mechanism to lock the head assembly 300 at desired orientation. The location of joystick 908 is designed ergonomically in such a way that that surgeons can easily use it using thumb and lock it with just by releasing the joystick 908.

The bottom surface of the handle 200 contains two gun type triggers, i.e., one main trigger 208 and one small trigger 210. The main trigger can be used to drive a needle of a suturing unit incorporated in the head assembly 300, and the small trigger 210 can be used for any one of grasping, cutting, cauterization and knot tightening, after the function shifter button 204 has been moved to the appropriate position.

The right surface of the handle 200 can include a scissors activation button 222 (refer to FIG. 1B), which can be a sliding button. Sliding the scissors activation button 222 to a scissors position can convert the grasper units of the head assembly to a scissor by projecting blades out of a slit in the grasping surface of the graspers of the grasper units. Now, by actuating the grasper a tissue can be cut in place of grasping. Further, when the function shifter button 204 is in a cautery position, the blades can receive an AC or DC current for cauterization.

The left surface of the handle 200 incorporates a grasper activation knob 212L for lower grasper unit and the right surface of the handle 200 includes a grasper activation knob 212R for upper grasper unit. The grasper activation knobs 212L and 212R are configured as toggle buttons, wherein a first pressing of the grasper activation knob 212L/212R (individually/collectively referred to as grasper activation knob(s) 212) engages a driving means, such as a motor 218, to the corresponding grasper unit and second pressing of the grasper activation knob 212 disengages the motor 218 from the corresponding grasper unit. The grasper activation knobs 212 can also be rotated manually to fine tune the grasping force. When the motor is engaged to the selected grasper unit and the function shifter button 204 has been moved to the grasping position, pressing of small trigger shall actuate the corresponding grasper unit to grasp the tissue or thread as the case may be.

The grasper activation knobs 212 have an inbuilt locking mechanism to retain the corresponding grasper unit in the grasping position, and pulling the grasper activation knob 212 out unlocks the corresponding grasper unit and the graspers release the grasped tissue or the suturing thread by moving to open position under biasing force on the graspers of the grasper unit.

The top surface of the handle 200 includes a knot tightening activation knob 214 that functions in same manner as the grasper activation knob 212. Specifically, when the knot tightening activation knob 214 is pressed, the motor 218 gets coupled to both upper and lower grasper units to move both the graspers of both the grasper units perpendicular to the planes of the grasper units to pull the grasped threads for tightening the knot.

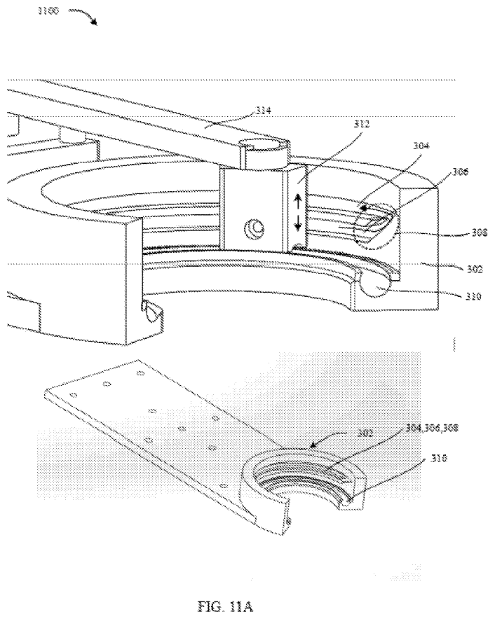

As shown in FIG. 2, and further in FIG. 10, each of the grasper activation knobs 212 and the knot tightening activation knob 214 is coupled to a bevel gear, such as bevel gears 1002-1, 1002-2, and 1002-3, which, when the corresponding knob 212/214 is pressed, move inwards to engage with an input bevel gear 1004 coupled to the motor 218. The coupling of the bevel gears 1002 to the input bevel gear 1004 results in transfer of rotation of the motor 218 to a corresponding pulley, such as first pulleys 1006-1 and 1006-2 coupled to the grasper activation knobs 212 and the second pulley 1006-3 coupled to the knot tightening activation knob (hereinafter, individually/collectively referred to as pulley(s) 1006). The pulleys 1106 have respective ropes wound around them, which, on being pulled due to rotation of the pulley 1006, actuate the grasper units for the corresponding functionality.

The main trigger 208 can be coupled to a suturing pulley 216 such that when the main trigger 208 is pressed, the suturing pulley 216 rotates to pull a suturing rope 220 wound around the suturing pulley 216. The suturing rope 220 can be coupled to a driving mechanism of the suturing unit of the head assembly 300. The main trigger 208, the suturing pulley 216 and the driving mechanism of the suturing unit can be configured such that one full pressing of the main trigger 208 results in 180 degree turn of the suturing needle of the suturing unit. A second pressing of the main trigger 208, after it has been released from the first pressing to come back to the starting/released position, can make the needle turn by additional 180 degrees, making a full turn of the needle. In different embodiments, it is possible to provide mechanized means for actuating the suturing unit, wherein motor 218 can be used to drive a pulley to pull the suturing rope 220 in the same manner as for the grasping units.

FIG. 3 shows an exploded view of the head assembly 300 having a suturing unit 1100/1400, an upper grasper unit, such as grasper unit 400-1, located in a plane above the suturing unit 1100/1400, and a lower grasper unit, such as grasper unit 400-2 (hereinafter, individually/collectively referred to as grasper unit(s) 400) located in a plane below the suturing unit 1100/1400. The two grasper units 400 can be generally similar in construction, i.e., one can be mirror image of the other.

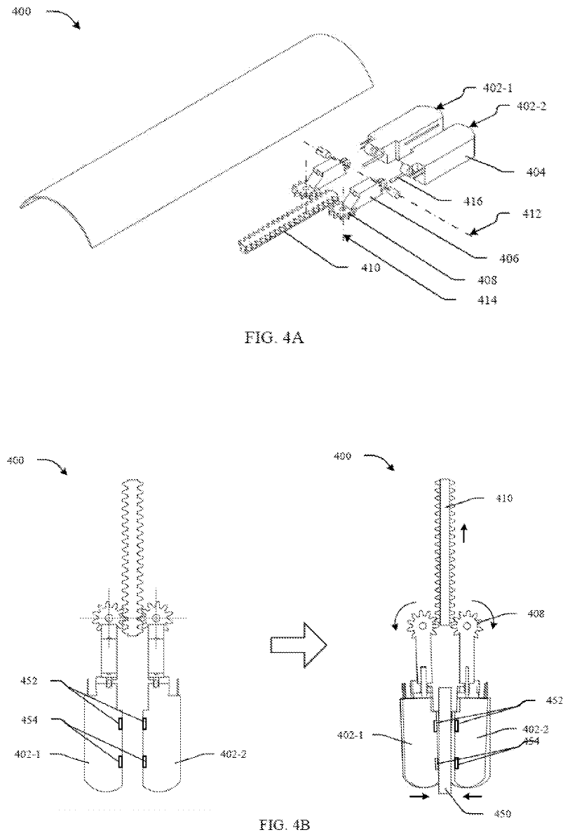

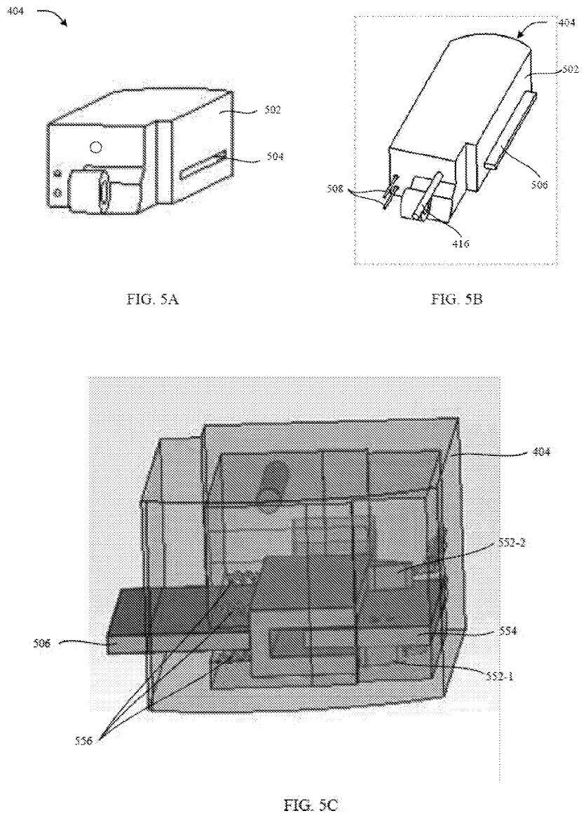

FIG. 4A shows an exploded view of the grasper unit 400, which can include a pair of graspers, such as grasper 402-1 and grasper 402-2 (individually/collectively referred to as grasper(s) 402). Each grasper 402 can include a front body 404 and a rear body 406. The front body 404 can be pivotally coupled to the rear body 406, such as by a hinge joint, for rotation about an axis 412 that is parallel to the plane of the corresponding grasper unit 400. The rear body 406 of each grasper 402 can be coupled to a pinion 408 such that rotation of the pinion 408 results in pivotal movement of the grasper 402 about an axis 414 that is perpendicular to the plane of the grasper unit 400.

In accordance with an embodiment, each of the grasper units 400 can include a double sided rack 410. The two sides of the rack can be in engagement with the pinions 408 of the two graspers 402 of the grasper unit 400 such that pulling of the rack 410 towards the proximal side results in the two graspers 402 to move towards each other for grasping a tissue 450, as shown in FIG. 4B, or a suturing thread.

The racks 410 of each of the grasper units can be coupled to the corresponding first pulleys 1006-1 and 1006-2 of the motor and gear drive mechanism 1000 (refer FIG. 10) in the handle 200 by a grasper rope such that rotation of the pulley causes the grasper rope to be pulled to move the graspers 402 to grasp a tissue or a suturing thread.

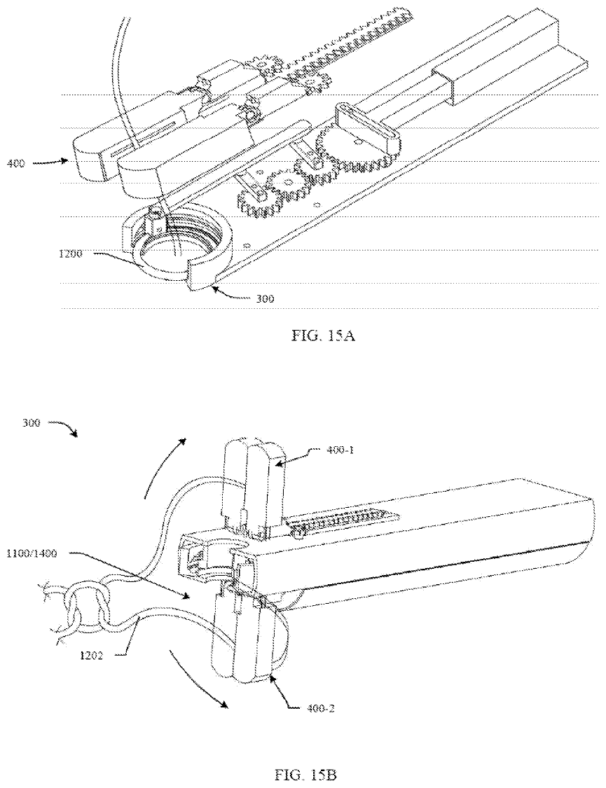

In accordance with an embodiment, the front bodies 404 of the two graspers of each of the grasper units 400 can be coupled to a second pulley 1006-3 (refer FIG. 10) in the handle 200 by a knot pulling rope 416 such that rotation of the second pulley 1006-3 causes the knot pulling ropes 416 to be pulled to move the front body of the graspers of the two grasper units, perpendicular to the corresponding planes in direction away from each other for pulling two ends of the suturing thread for knot tightening, as shown in FIG. 15B.

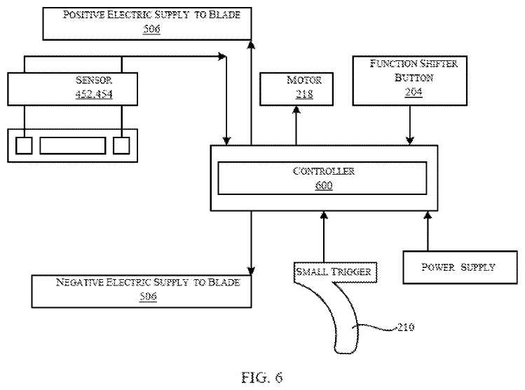

In accordance with an embodiment, the graspers unit 400 can include a set of infrared sensors 452 and pressure sensors 454 located on the grasping surface of the graspers 402 as shown in FIG. 5B. The pair of infrared sensors 452 placed on the opposed graspers 402 can be operatively coupled to a controller of the device to determine thickness of the grasped tissue 450. One of the two infrared sensors 452 provided on the opposing surfaces of two graspers 402 of the grasper unit 400 can be an emitting sensor and other can be a receiving sensor that receives the infrared radiation emitted by the emitting sensor after they have passed through the grasped tissue 450. Loss in strength of the infrared radiation due to passing through the tissue 450 can be used by the controller to estimate thickness to the grasped tissue. The estimated thickness of the issue 450 can be used by the controller for controlled actuation of the motor 218 during grasping.

Furthermore, the pressure sensors 454 can be provided at tip of the graspers 402, on the front body 404 of the graspers 402 to be precise, to give feedback to the controller while the grasper unit 400 is being actuated. To be specific, the controller can stop further rotation of the motor 218 when the sensed pressure exceeds a predefined pressure value. This can prevent damage to the grasped tissue 450 during grasping due to crushing. However, if, for any reason, the user feels that the tissue needs to be grasped with a higher pressure, he can rotate the corresponding grasper activation knob 212 to increase the grasping pressure on the grasped tissue 450.

In accordance with an embodiment, the infrared sensors 452 can also be used to detect presence of a blood vessel in the grasped tissue. This can be of importance when the grasper unit 400 is being used as a scissors or cautery. The controller, coupled to the infrared sensors 452, can be configured to detect flow of blood in the grasped tissue 450. Pulsation in the determined blood flow value can be interpreted as presence of a blood vessel. The controller, on detection of a blood vessel, can issue a warning to the user, such as by a buzzer. The arrangement of the infrared sensors 452 can also be used to differentiate between a normal tissue and a diseased tissue in which there is decreased perfusion of blood.

FIGS. 5A-5C show features of a front body 404 of a grasper 402 of the grasper units 400, which feature enable conversion of the grasper unit 400 to a scissors or cautery. As shown in FIG. 5A, the front body 404 can include a slit 504 located on a grasping surface 502 of the front body 404. FIG. 5B shows a blade 506, which is slidably configured within the front body 404, projected out of the slit 504. The blade 506 can be made to project out of the slit 504 by actuating a pair of cams, such as cams 552-1 and 552-2 (collectively referred to as cams 552), shown in FIG. 5C. The cams 552 can be pivotally fixed on a guide plate 554, one cam 552 on an upper surface of the guide plate 554 and the other cam 552 located on a lower surface of the guide plate 554, and can be in engagement with two legs of the Y-shaped blade 506 with the guide plate located therebetween, as shown in FIG. 5C. There can be springs 556 to bias the blade 506 inwards to a retracted position.

The cams 552 can be coupled to the scissors activation button 222 on the handle 200 through one or more scissors ropes 508, shown in FIG. 5B. As can be understood, blades 506 of both graspers 402 have to project out for the grasper unit 400 to work as a scissors, and accordingly scissors ropes of both the graspers 402 have to be coupled to the scissors activation button 222 for simultaneous pulling.

In accordance with an embodiment, the blades 506 can also be connected to a current source for the grasper unit 400 to work as a cautery. The functionality can be activated by shifting the function shifter button 204 to the cautery mode, after which when the small trigger 210 is pressed, the controller completes a circuit through the blades 506 of the grasper unit 400 for an AC or DC current to pass through the tissue held therebetween.

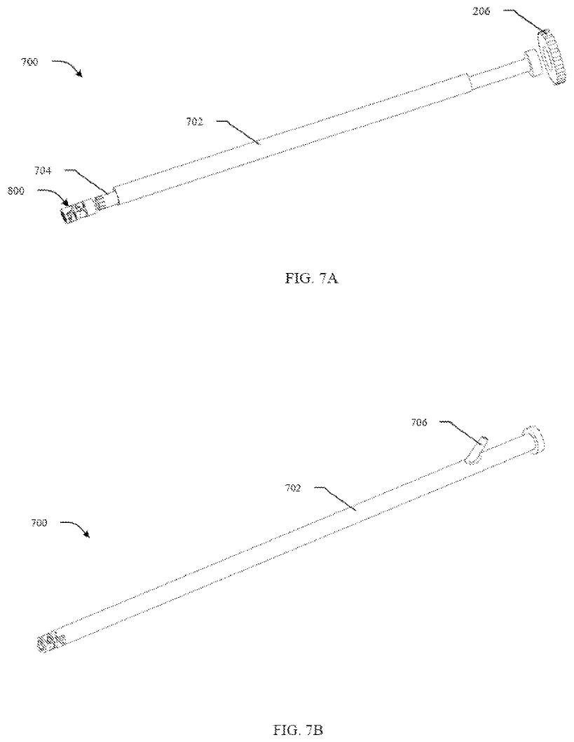

FIG. 6 is a block diagram of a control mechanism of the device 100 showing different elements that enable different functionalities of the device. As shown a controller 600 of the device can be operatively coupled to the function shifter button 204, the small trigger 210 and the motor 218 provides on the handle 200. The controller can also be operatively coupled to the sensors 452, 454 and blades 506 on the grasper unit 400. The controller can be embodied in an electric circuit board, which can also include a processor and a memory. The memory can store look up tables, such as for estimating thickness of the grasped tissue based on the infrared sensor 452 signals. Based on position of the function shifter button 204, the controller 600 can, on pressing of the small trigger 210, actuate the appropriate elements of the device to achieve the selected functionality. For example, when the function shifter button 204 is in cautery mode, the controller 600 can on pressing of the small trigger 210 can supply current to the blades 506 of the grasper unit 400 for cauterization. Similarly, when the function shifter button 204 is in grasping mode, the controller can take feedback from the infrared sensors 452 to determine thickness of the tissue between the graspers of the activated grasper unit 400 and activate the motor 218 to make it rotate by a required angle of rotation. The controller can also take feedback from the pressure sensors 454 to stop rotation of the motor 218 when the sensed pressure exceeds a predefined pressure value to prevent damage to the tissue 450 due to crushing.

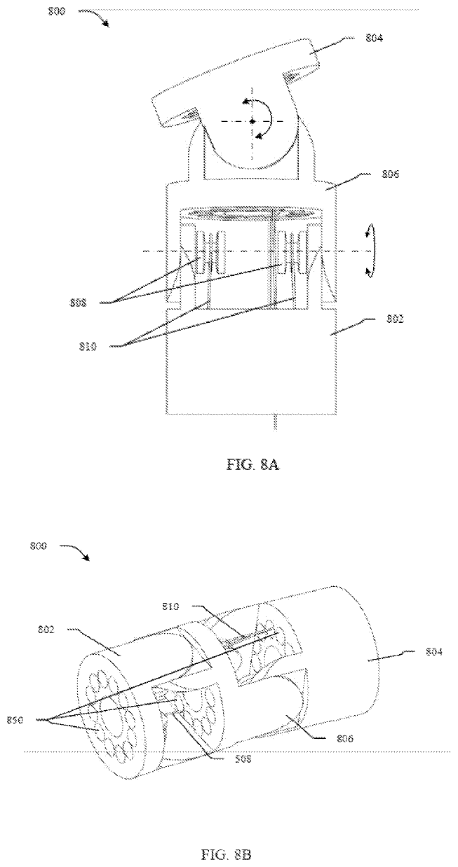

FIGS. 7A and 7B show two different views of the shaft 700. The shaft 700 can include an outer casing 702 and an inner tube 704 located coaxially within the outer casing 702. A proximal end of the inner tube 704 can be fixed to the rotating knob 206 and the distal end can be fixed to a proximal end of the articulation joint 800. The inner tube 704 can carry various ropes from the handle to the head assembly 300 through the articulation joint 800. Annular space between the outer casing 702 and the inner tube 704 can be used for feeding an irrigation fluid to abdominal cavity of the patient under surgery. The irrigation fluid can be fed through an irrigation port 706 provided on the outer casing 702, as shown in FIG. 7B.

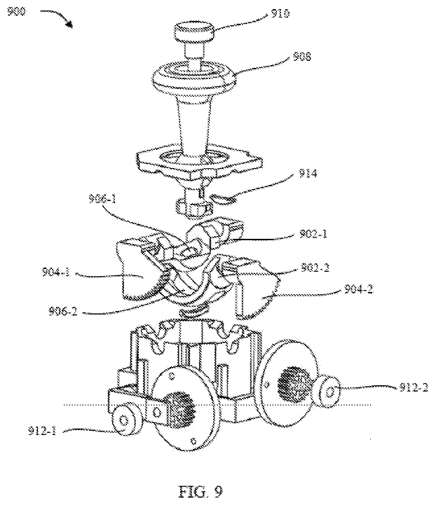

FIGS. 8A and 8B show top view and perspective view respectively of the articulation joint 800. The articulation joint 800 can include a first yoke 802, a second yoke 804 and an intermediate yoke 806 coupling the first yoke 802 to the second yoke 804, each by a pivotal joint. Axes of the two pivot are mutually perpendicular to provide two degrees of freedom in two mutually perpendicular axes. Each pivot axis can have pulleys, such as pulleys 808, to couple the articulation joint 800 to the manipulator 900 through manipulator ropes 810.

As shown in FIG. 8B, the yokes 802, 804 and 806 can include holes 850 for passage of different ropes, such as scissors ropes 508, as shown therein, from the shaft 700 to the head assembly 300.

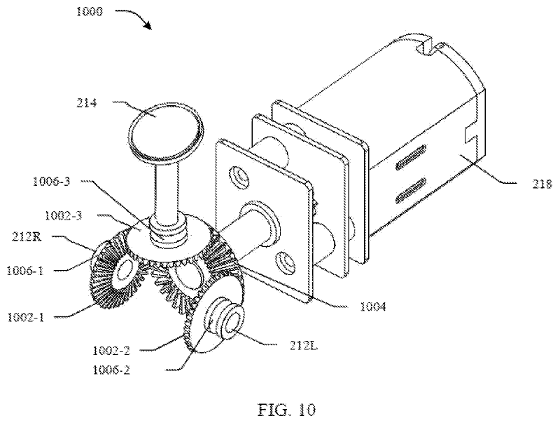

FIG. 9 shows an exploded view of the manipulator 900. The manipulator includes two curved gimbals, such as gimbals 902-1 and 902-2 (collectively and individually referred to as gimbal(s) 902), placed one over the other for sliding movement. There can be a slot in each gimbal 902, such as slots 906-1 in the gimbal 902-1 and slot 906-2 in gimbal 902-2 (collectively and individually referred to as slot (s) 906). A joystick 908 engages with the slots 906 to move the gimbals 902 in direction of slots 906. The gimbals incorporate gears 904-1 and 904-2 that are in engagement with corresponding gears to which pulleys 912-1 and 912-2 (collectively and individually pulley(s) 912, hereinafter) is coupled. The pulleys can carry manipulator ropes 810 to couple the manipulator 900 to the articulation joint 800.

The manipulator includes a lock button 910 coupled to the joystick 908 telescopically. A lower end of the lock button that projects out of the joystick 908 includes a stopper 914 that rests against the lower gimbal 902. A lower end of the joystick 908 also carries a stopper that engages with the upper gimbal 902. The lock button is biased in upward direction, which causes the two stoppers 914 to press the gimbals 902 against each other to lock the gimbals, and thereby the manipulator, in any position. Pressing of the lock button 910 causes the stoppers 914 to move away from the gimbals 902 thereby allowing the gimbals 902 to move relative to each other. Movement of the gimbals 902 is transferred to the respective pulleys 912 through the respective gears 904.

FIG. 10 is a depiction of the motor and gear drive mechanism 1000 for moving the graspers 402 of the two grasper units 400 during grasping and knot tightening operation, which functions as explained earlier.

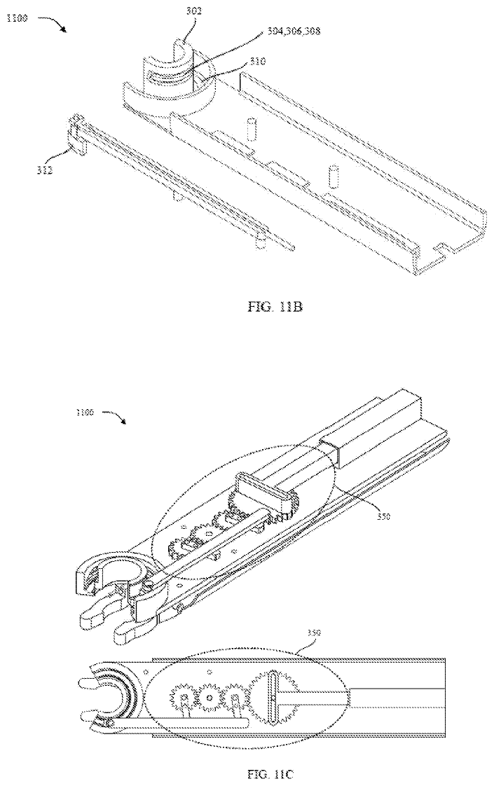

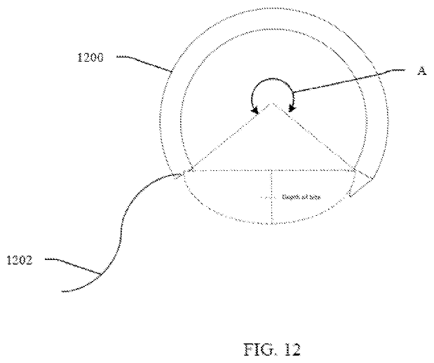

FIGS. 11A to 11C show details of an embodiment of the suturing unit of the head assembly 300. The suturing unit 1100 for minimally invasive surgery can include a housing 302 having a pair of parallelly disposed more than half circle tracks, comprising an upper track 304 and a lower track 306, and a needle track 310. The needle track 310 can be configured to accommodate a suturing needle, such as suturing needle 1200 (also referred to simply as needle 1200 and the two terms used interchangeably herein), shown in FIG. 12, for movement of the suturing needle 1200 along the needle track 310. The upper track 304, the lower track 306 and the needle track 310 can be coaxially arranged on different surfaces of the housing 302. For example, the upper track 304 and the lower track 306 can be on an inner circumference of the housing 302 and the needle track 310 can be on a flat surface that is perpendicular to an axis of the upper and lower tracks 304 and 306, as shown in FIGS. 11A and 11C. Alternatively, as shown in FIG. 1113, the upper track 304 and the lower track 306 can be on an outer circumference of the housing 302 and the needle track 310 can be on a flat surface that is perpendicular to an axis of the upper and lower tracks 304 and 306.

The suturing unit 1100 can further include a puck 312 in engagement with one of the upper track 304 and the lower track 306 at any given time. The puck 312, when in engagement with the lower track 306, can be in engagement with, and press against, the suturing needle 1200 such that movement of the puck 312 results in movement of the suturing needle 1200 along the needle track 310, and when the puck 312 is in engagement with the upper track 304, the puck 312 is lifted off the suturing needle 1200 and moves without imparting movement to the suturing needle 1200.

The suturing unit 1100 can further include a driving mechanism, such as driving mechanism 350 shown in FIG. 11C to move the puck 312 along the upper track 304 and the lower track 306. A link 314 of the driving mechanism 350 can be coupled to the puck 312 for driving the puck 312.

The upper track 304 and the lower track 306 can be connected to each other at their two ends through a slanted track, such as the slanted track 308 shown in FIG. 11A, such that when the puck 312 is moved to the end of the lower track 306 the puck 312 shifts upward to get engaged to the upper track 304, and when the puck 312 is moved in reverse direction to the end of the upper track 304 the puck 312 shifts downward to get engaged to the lower track 306. The driving mechanism 350 can be coupled to the main trigger 208 through the suturing rope 220.

In an embodiment, the driving mechanism 350 can be a Scotch yoke mechanism, shown in FIG. 11C. However, other mechanisms are well within the scope of the present disclosure.

FIG. 12 illustrates an exemplary view of the suturing needle 1200 used with the suturing unit. The suturing needle 1200 can be a more than half circle needle having a subtended angle A in the range of 240-270 degrees. In preferred embodiments, the subtended angle A of the suturing needle 1200 can be 240 degrees, or 250 degrees or, 260 degrees, or 270 degrees. The suturing needle can have sharp pointed leading end and a trailing end. A suturing thread 1202 can be fixed to the trailing end of the suturing needle 1200.

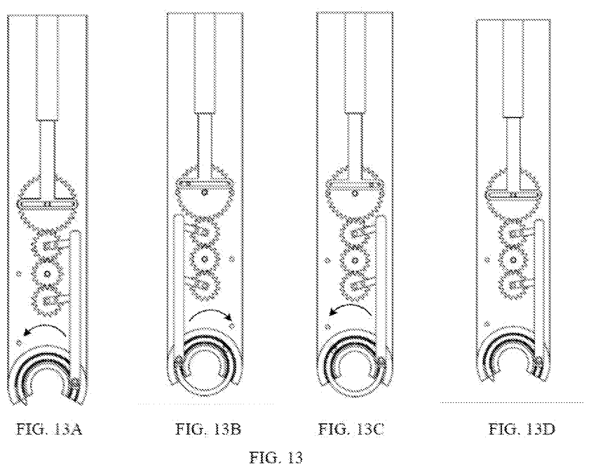

FIGS. 13A to 13D show functioning of a suturing unit. Starting from a start position of the suturing needle 1200 shown in FIG. 13A, where the puck 312 is engaged with the lower track 306, when a user presses the main trigger 208, the coupled suturing pulley 216 rotates and pulls the suturing rope 220 to drive the driving mechanism 350. The suturing needle 1200 undergoes 180 degrees rotation, as shown in FIG. 13B, as a result of one pressing of the main trigger 208. At this point, the puck 312 reaches the end of the lower track 306 and shifts to the upper track 304. Now when the mail trigger is released, the puck 312 moves in opposite direction in engagement with the upper track 304 without driving the needle 1200. Therefore needle remains in same position as shown in FIG. 13C while the puck 312 reaches the end of the upper track 304 and shifts back to the lower track 306. A second pressing of the main trigger 208 now results in the suturing needle 1200 rotating by an additional 180 degrees completing a full circle, and release of the main trigger 208 can bring the puck 312 back to the starting position, as shown in FIG. 13D.

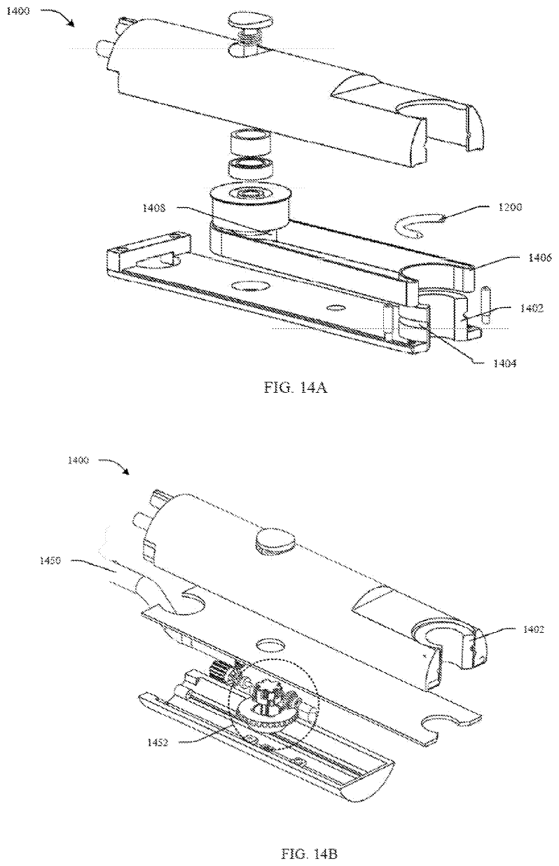

In an alternative embodiment of the suturing unit shown in FIGS. 14A and 14B, the suturing unit 1400 can be based on the suturing needle 1200 being driven by a belt instead of a puck, as in the suturing unit 1100. The suturing unit 1400 can include a housing 1402 having a needle track 1404 configured on an outer periphery of the housing 1402. A suturing needle 1200 can be positioned in the needle track 1404 for movement along the needle track 1404. The suturing unit can further include a belt 1406 configured around the outer periphery of the housing 1402 such that the belt 1406 presses against the suturing needle 1200 to move the needle 1200 along the needle track 1404 as the belt 1406 moves. There can be a plurality of pulleys, such as pulley 1408, to move the belt 1406 around the outer periphery of the housing 1402. In an aspect, one of the plurality of pulleys, such as pulley 1408, can be is coupled to a motor (not shown here) by a flexible rotary shaft 1450 shown in IG 14B, for driving the belt 1406. The suturing unit 1400 can be mechanized driven by a motor in place of manual by pressing the main trigger 208.

FIGS. 15A and 15B are views of the head assembly respectively showing knotting and knot tightening. After the needle 1200 has taken a bite of a grasped tissue, the grasped tissue can be released by releasing the grasper units, such as by pulling the grasper activation knobs 212 outwards. Now the head assembly 300 can be manipulated to grasp a free end portion of the suturing thread 1202 by one of the upper or lower grasper units 400, which, on account of configuration of the head assembly 300, would result in the loose end portion of the suturing thread 1202 to be located within a circular zone defined by movement of the needle 1200. Now the needle 1200 can be moved by one full turn, i.e., by 360 degrees, or more depending on type of knot required, to wind the suturing thread around the free end portion of the suturing thread to take one loop and repeat according to type of knot. Thereafter, other grasper unit 400 can be used to grasp a needle side portion of the suturing thread 1202. Once both sides of the suturing thread 1202 are grasped by the two grasper units 400, the two grasper units 400 can be actuated for movement perpendicular to the planes of the grasper units 400 to pull the respective ends of the suturing thread 1202, as shown in FIG. 15B, to tighten the knot. In particular, the knot tightening can be activated by pushing the knot tightening activation knob 214 and thereafter pressing the small trigger 210.

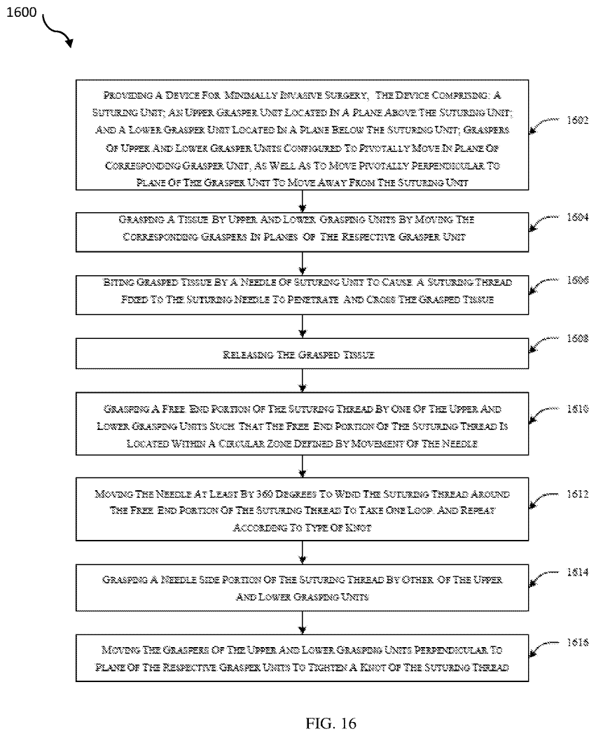

FIG. 16 is a method flow diagram for the method for suturing, knotting and knot tightening using the head assembly 300 of the disclosed device 100. The method 1600 can include, at step 1602, providing a device, such as device 100 shown in FIGS. 1 to 15B, for minimally invasive surgery. The method 1600 can, at step 1604, involve grasping the tissue, such as tissue 450 shown in FIG. 4B, by an upper grasper unit and a lower grasper unit, such as grasper units 400 shown in FIGS. 4A and 4B, by moving the corresponding graspers, such as graspers 402 shown in FIG. 4A, in the planes of the respective grasper unit 400. The step 1606 of the method 1600 can be to bite the grasped tissue by a needle, such as needle 1200 shown in FIG. 12, of a suturing unit, such as a suturing unit 1100 or 1400 shown in FIGS. 11A-11C and FIGS. 14A and 14B respectively, to cause a suturing thread, such as thread 1202 shown in FIG. 12, fixed to the suturing needle 1200 to penetrate and cross the tissue 450.

Step 1608 of the method 1600 can be to release the grasped tissue 450, and step 1610 can be to grasp a free end portion of the suturing thread 1202 by one of the upper and lower grasper units 400 such that the free end portion of the suturing thread is located within a circular zone defined by the movement of the needle 1200.

Step 1612 of the method 1600 can be to move the needle 1200, as shown in FIG. 15A, at least by 360 degrees to wind the suturing thread around the free end portion of the suturing thread to take one loop and repeat according to type of knot. At step 1614, the method 1600 can involve grasping a needle side portion of the suturing thread 1202 by other of the upper and lower grasper units 400, and at step 1616 the graspers 402 of the two grasper units 400 can be moved perpendicular to the plane of the respective grasper units 400 to tighten a knot of the suturing thread 1202.

Thus the present disclosure provides a multifunctional device 100 for minimally invasive surgery that includes functionalities of grasping, cutting, cauterizing, suturing, knotting and knot tightening, among others, during laparoscopic surgery. Thus the disclosed device 100 overcomes drawbacks of the conventional procedures for minimally invasive surgery that require multiple devices, and thereby eliminating need for multiple instruments and multiple grasps and release movements during the process. As various functionalities are mechanized, i.e., motor driven, device can be modified for integration with surgical robotics as well after modifications that would be obvious to those skilled in the art.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present disclosure provides an improved laparoscopic surgical device that overcomes the limitations of conventional methodologies for grasping suturing, knotting &knot tightening during minimally invasive surgeries.

The present disclosure provides a single device for laparoscopic surgery that facilitates grasping, cutting, cauterizing, suturing, knotting and knot tightening, among others, during laparoscopic surgery.

The present disclosure provides a laparoscopic surgical device that can perform most laparoscopic functions using a single instrument, thereby eliminating the need to switch instruments for each function.

The present disclosure provides a laparoscopic surgical device that includes feedback mechanism during grasping of tissues for control of force.

The present disclosure provides a laparoscopic surgical device that includes mechanism for detecting presence of a blood vessel in the grasped tissues to prevent accidental damage to the blood vessel.

The present disclosure provides a laparoscopic surgical device that incorporates 6 degrees of freedom in space for movements of a head assembly.

The present disclosure provides a laparoscopic surgical device that automates the whole process of intracorporeal suturing, knotting, and knot tightening into a single process.

The present disclosure provides a laparoscopic surgical device that avoids multiple grasps and release movements during the process of intracorporeal suturing.

The present disclosure provides a laparoscopic surgical device that can be easily incorporated with surgical robotics systems.

The present disclosure provides a laparoscopic device which can assist the surgeons in single incision laparoscopic surgeries.

We claim:

1. A multifunctional device for minimally invasive surgery, comprising:
a handle;
a shaft coupled to a distal end of the handle;
a multifunctional head assembly configured at the distal end of the shaft through an articulation joint such that the articulation joint allows the head assembly to pivotally move relative to the shaft in two degrees of freedom; wherein the head assembly comprises;
a suturing unit;
an upper grasper unit located in a plane above the suturing unit; and
a lower grasper unit located in a plane below the suturing unit;
wherein each of the upper and lower grasper units comprises a pair of graspers, the graspers are configured to pivotally move in the plane of the corresponding grasper unit to grasp tissues or a suturing thread, as well as to move pivotally perpendicular to the plane of the grasper unit to pull the grasped suturing thread for knot tightening.

2. The device as claimed in claim 1, wherein the graspers of the at least one of the upper and lower grasper units comprises a cutting blade configured within the grasper for sliding movement such that in an actuated position a cutting edge of the cutting blade projects out of a slit on a grasping face of the grasper for use of the grasper unit as a scissors.

3. The device as claimed in claim 2, wherein the graspers comprise a set of cams in engagement with the corresponding blade, the set of cams coupled by scissors ropes to a scissors activation button provided on the handle such that moving the scissors activation button to a cutting mode position pulls the scissors rope to rotate the set of cams to cause the blades to project out of the slits, and wherein moving the scissors activation button out of the cutting mode position releases the scissors rope to move the set of cams back thereby allowing a set of springs to move the blades within the slits.

4. The device as claimed in claim 2, wherein the blades are configured to get any of a direct and alternating current for the blades to function as cautery.

5. The device as claimed in claim 1, wherein the shaft comprises an outer tubular casing and an inner tube coupled to the head assembly through the articulation joint.

6. The device as claimed in claim 5, wherein the inner tube is coupled to a rotating knob located at a distal end of the handle such that rotation of the rotating knob by a user results in rotation of the head assembly about a longitudinal axis of the shaft.

7. The device as claimed in claim 6, wherein the articulation joint is coupled to a manipulator through a pair of ropes, the manipulator being located on the handle such that a joystick of the manipulator is operable by a thumb of a user; wherein movement of the joystick laterally and in up and down direction results in the articulation joint pivotally moving the head assembly about the two mutually perpendicular axes, each perpendicular to the longitudinal axis of the shaft, thereby providing three degrees of rotational freedom to the head assembly.

8. The device as claimed in claim 7, wherein the manipulator comprises an upper gimbal and a lower gimbal placed over one another and coupled with the joystick for pivotal motion about two mutually perpendicular axes, each gimbal coupled to a pulley for the respective ropes that couple the manipulator to the articulation joint.

9. The device as claimed in claim 8, wherein the manipulator comprises a lock button, the lock button configured in combination with the joystick to press a set of stoppers against the gimbals to force the gimbals against each other to lock the manipulator in any position, and wherein pressing the lock button separates the stoppers from the gimbals to allow movement of the gimbals for manipulating the articulation joint.

10. The device as claimed in claim 1, wherein the each grasper comprises a front body and a rear body, the front body pivotally coupled to the rear body for rotation about an axis that is parallel to the plane of the corresponding grasper unit, and wherein the rear body is coupled to a pinion such that rotation of the pinion results in pivotal movement of the grasper about an axis perpendicular to the plane of the corresponding grasper unit.

11. The device as claimed in claim 10, wherein each of the grasper units comprises a rack in engagement with the pinions of the corresponding graspers such that pulling of the rack towards the proximal side results in the two graspers to move towards each other for grasping a tissue or a suturing thread.

12. The device as claimed in claim 11, wherein the racks of each of the grasper units is coupled to a first pulley in the handle by a first rope such that rotation of the pulley causes the rope to be pulled to move the graspers to grasp a tissue or a suturing thread.

13. The device as claimed in claim 12, wherein the front bodies of the two graspers of each of the grasper units is coupled to a second pulley in the handle by a second rope such that rotation of the second pulley causes the second ropes to be pulled to move the graspers of the two grasper units perpendicular to the corresponding planes in direction away from each other for pulling two ends of the suturing thread for knot tightening.

14. The device as claimed in claim 13, wherein the two first pulleys and the second pulley are coupled to a corresponding bevel gear and a corresponding grasper activation knob and a knot tightening activation knob respectively, wherein the activation knobs have a cam mechanism such that a first pressing of the activation knob causes the corresponding bevel gear to move inwards to engage with an input bevel gear coupled to a motor, and a second pressing of the activation knob results in the bevel gear to move outwards to disengage from the input bevel gear.

15. The device as claimed in claim 14, wherein the activation knobs include an inbuilt locking mechanism to lock the corresponding pulley, and thereby the corresponding grasper unit in an actuated position, and wherein the locking mechanism is configured such that, pulling the activation knob outwards unlocks the pulley and the grasper unit to return the graspers to corresponding resting positions.

16. The device as claimed in claim 14, wherein the upper and lower grasper units comprise a set of infrared sensors and pressure sensors configured on the graspers.

17. The device as claimed in claim 16, wherein the device comprises a controller operatively coupled to the set of infrared sensors, the pressure sensors and to the motor, the controller being configured to provide, based on signals from the infrared sensors, a feedback on thickness of the tissue being grasped, and further configured to stop, based on feedback from the pressure sensors, further rotation of the motor when the sensed pressure exceeds a predefined pressure value.

18. The device as claimed in claim 17, wherein the controller is configured to detect presence of a blood vessel in the grasped tissue based on pulsation in the signal from the infrared sensors, the signal being indicative of flow of blood in the grasped tissue; and further configured to issue a warning to a user of the device, if a blood vessel is detected.

19. The device as claimed in claim 1, wherein the handle comprises a main trigger for actuating the suturing unit, the main trigger being coupled to a suturing pulley such that when the main trigger is pressed, the suturing pulley rotates to pull a suturing rope wound around the suturing pulley, the suturing rope being coupled to an driving mechanism of the suturing unit; and wherein the main trigger, the suturing pulley and the driving mechanism of the suturing unit are configured such that one full pressing of the main trigger results in 180 degree turn of the suturing needle of the suturing unit, and a second pressing of the main trigger, after the main trigger has been released from the first pressing to come back to the starting position, makes the suturing needle turn by additional 180 degrees, making a full turn of the suturing needle.

20. The device as claimed in claim 1, wherein the handle comprises a function shifter button and a small trigger operatively coupled to a controller of the device, wherein the function shifter button is configured for selecting between grasping, cutting, knot tightening, cautery and suturing operations, and wherein the controller activates the selected function based on the position of the function shifter button, when the small trigger is pressed.

21. A multifunctional device for minimally invasive surgery, comprising:
a handle;
a shaft coupled to a distal end of the handle;
a head assembly configured at the distal end of the shaft; the head assembly comprising at least one grasper unit, the grasper unit comprising a pair of graspers to grasp at least a tissue;
wherein each of the graspers of the at least one grasper unit comprises a cutting blade configured within the grasper for sliding movement such that in an actuated position a cutting edge of the cutting blade projects out of a slit on a grasping face of the grasper for use of the grasper unit as a scissors.

22. The device as claimed in claim 21, wherein the blades are configured to get flow of any of a direct and alternating current for the blades to function as cautery.

23. The device as claimed in claim 21, wherein the at least one grasper unit comprises a set of infrared sensors, and the device comprises a controller to detect presence of a blood vessel in the grasped tissue based on pulsation in the signal from the infrared sensors, the signal being indicative of presence of blood in the grasped tissue; and the controller further configured to issue a warning to a user of the device, if a blood vessel is detected.

24. A multifunctional device for minimally invasive surgery, comprising:
a handle;
a shaft coupled to a distal end of the handle;
a head assembly configured at the distal end of the shaft; the head assembly comprising at least one grasper unit, the grasper unit comprising a pair of graspers to grasp at least a tissue;
wherein the grasper units comprise a set of infrared sensors and pressure sensors configured on the graspers to provide a feedback on thickness of the tissue being grasped and pressure applied on the tissue during grasping to control actuation of the grasper units to prevent crushing of the grasped tissue.

25. A multifunctional device for minimally invasive surgery, comprising:
a handle;
a shaft coupled to a distal end of the handle;
a head assembly configured at the distal end of the shaft; the head assembly comprising at least one grasper unit, the grasper unit comprising a pair of graspers to grasp at least a tissue;
wherein the at least one grasper unit comprise a set of infrared sensors configured on the graspers; and
wherein the device comprises a controller to detect presence of a blood vessel in the grasped tissue based on pulsation in the signal from the infrared sensors, the signal being indicative of presence of blood in the grasped tissue; and the controller further configured to issue a warning to a user of the device, if a blood vessel is detected.

26. A method for suturing, knotting and knot tightening a tissue during a minimally invasive surgery, comprising the steps of:
providing a device for minimally invasive surgery, the device comprising: a suturing unit; an upper grasper unit located in a plane above the suturing unit; and a lower grasper unit located in a plane below the suturing unit; wherein graspers of each of the upper and lower grasper units are configured to pivotally move in the plane of the corresponding grasper unit, as well as to move pivotally perpendicular to the plane of the grasper unit to move away from the suturing unit;
grasping the tissue by an upper grasper unit and a lower grasper unit by moving the corresponding graspers in the planes of the respective grasper unit;
biting the grasped tissue by a suturing needle of the suturing unit to cause a suturing thread fixed to the suturing needle to penetrate and cross the tissue;
releasing the grasped tissue;
grasping a free end portion of the suturing thread by one of the upper and lower grasper units such that the free end portion of the suturing thread is located within a circular zone defined by movement of the suturing needle;
moving the suturing needle at least by 360 degrees to wind the suturing thread around the free end portion of the suturing thread to take one loop and repeat according to type of, knot;
grasping a needle side portion of the suturing thread by other of the upper and lower grasper units; and
moving the graspers of the upper and lower grasper units perpendicular to the plane of the respective grasper units to tighten a knot of the suturing thread.

27. A suturing unit for minimally invasive surgery, comprising
a housing having a pair of more than half circle tracks, comprising an upper track and a lower track, and a needle track;
a suturing needle positioned in the needle track for movement along the needle track;
a puck in engagement with the at least one of the upper track and the lower track, the puck, when in engagement with the lower track presses against the suturing needle being in engagement with the suturing needle such that movement of the puck results in movement of the suturing needle, and when in engagement with the upper track, the puck is lifted off the suturing needle and moves without imparting movement to the suturing needle; and
a driving mechanism to move the puck along the upper track and the lower track;
wherein the upper track and the lower track are connected to each other at their two ends such that when the puck is moved to the end of the lower track the puck shifts upward to get engaged to the upper track, and when the puck is moved in reverse direction to the end of the upper track the puck shifts downward to get engaged to the lower track.

28. The suturing unit as claimed in claim 27, wherein the driving mechanism is a Scotch yoke mechanism.

29. The suturing unit as claimed in claim 27, wherein the upper track, lower track and the needle track are configured on any of an inner periphery and an outer periphery of the housing.

30. A suturing unit for minimally invasive surgery, comprising;
a housing comprising a needle track configured on an outer periphery of the housing;
a suturing needle positioned in the needle track for movement along the needle track;
a belt configured around the outer periphery of the housing such that the belt presses against the suturing needle to move the suturing needle along the needle track as the belt moves; and
a plurality of pulleys to move the belt around the outer periphery of the housing;
wherein one of the plurality of pulleys is coupled to a motor by a flexible rotary shaft for driving the belt.
